(19) United States
(12) Patent Application Publication
(10) Pub. No.: US 2009/0081121 A1
(43) Pub. Date: Mar. 26, 2009

(54) LIPOSOME COMPOSITIONS USEFUL FOR TUMOR IMAGING AND TREATMENT

(75) Inventors: Gann Ting, Zhunan (TW); Yun-Long Tseng, Taipei City (TW); Jun-Jen Liu, Taipei City (TW); Hsin-Ell Wang, Taipei City (TW); Jeng-Jong Hwang, Taipei-City (TW); Yi-Ching Lu, Taipei City (TW); Wan-Chi Lee, Longtan Township (TW)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: National Health Research Institute, Zhunan (TW); National Yang Ming University, Taipei City (TW); Taiwan Liposome Company, Ltd., Taipei City (TW)

(21) Appl. No.: 12/237,878

(22) Filed: Sep. 25, 2008

Related U.S. Application Data
(60) Provisional application No. 60/975,309, filed on Sep. 26, 2007.

(51) Int. Cl. A61K 51/12; C07F 19/00

(52) U.S. Cl. 424/1.21; 534/10

(57) ABSTRACT

The invention relates to liposome compositions for delivering, for example, therapeutic, diagnostic, and imaging agents to a subject. Methods for preparing and using such liposome compositions are further provided. The compositions and methods of the invention find particular use in treating, diagnosing, and imaging a tumor in a subject.

### CROSS-REFERENCE TO RELATED APPLICATION

This application claims the benefit of U.S. Provisional Application Ser. No. 60/975,309, filed Sep. 26, 2007, which is herein incorporated by reference in its entirety.

### FIELD OF THE INVENTION

The present invention is generally related to the field of liposome compositions, particularly to liposome compositions for use in delivery of therapeutic and imaging agents to subjects in need thereof.

### BACKGROUND OF THE INVENTION

Liposomes, or lipid bilayer vesicles, have been used or proposed for use in a variety of applications in research, industry, and medicine, particularly for the use as carriers of diagnostic or therapeutic compounds in vivo (Lasic, Trends Biotechnol., 16: 307-321, 1998; Drummond et al., Pharmacol. Rev., 51: 691-743, 1999). Liposomes are usually characterized as microscopic vesicles having an interior aqua space sequestered from an outer medium by a membrane of one or more bilayers. Bilayer membranes of liposomes are typically formed by amphiphilic molecules, i.e., lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.).

A liposome typically serves as a carrier of an entity such as, without limitation, a chemical compound, a combination of compounds, or a radioisotope thereof, that is capable of having a useful property or exerting a useful activity. For this purpose, the liposomes are prepared to contain the desired entity in a liposome-incorporated form. The process of incorporation of a desired entity into a liposome is often referred to as “loading” (Lasic et al., FEBS Lett., 312: 255-258, 1992). The liposome-incorporated entity may be completely or partially located in the interior space of the liposome, within the bilayer membrane of the liposome, or associated with the exterior surface of the liposome membrane. The incorporation of entities into liposomes is also referred to as “encapsulation” or “entrapment”. The three terms “loading”, “encapsulation” and “entrapment” are used herein interchangeably to have the same meaning.

The purpose of incorporating an entity into a liposome is often to protect the entity from the destructive environment and rapid excretion while providing the opportunity for the encapsulated entity to exert the activity of the entity mostly at the site or in the environment where such activity is advantageous but less so at other sites where such activity may be useless or undesirable. This phenomenon is referred to as passive targeting delivery, especially to a desired site such as a neovascular or inflammatory site. For example, a radiopharmaceutical entrapped within a long-circulating liposome can be delivered to a tumor site to facilitate the diagnosis and/or treatment of the tumor. Moreover, this radiopharmaceutical formulation has a long duration in tumor sites and ascites to facilitate chemoradiotherapy.

Ideally, such liposomes can be prepared to include the desired entity, e.g., a compound or isotope, (i) with a high loading efficiency, i.e., high percentage of encapsulated entity relative to the total amount of the entity used in the encapsulation process, and (ii) in a stable form, i.e., with little release (i.e., leakage) of the encapsulated entity upon storage or generally before the liposome reaches the site or the environment where the liposome-entrapped entity is expected to exert its intended activity.

For therapeutics and radiopharmaceuticals, ideal radioisotopes are those with an abundance of low penetrating radiations, for example, beta emitters, alpha particle emitters, and auger electron emitters so that when the radiopharmaceuticals reach the disease target, the energy from the radioisotope is deposited at that site and does not irradiate nearby normal tissues. The energy of particles from different radioisotopes and their ranges in tissues will vary, as well as their half-life, and the most appropriate radioisotope will be different depending on the application, the disease and the accessibility of the disease tissue. Radiopharmaceuticals labeled with low-energy electron emitters, such as In-111, have several key advantages over traditional agents that emit higher-energy particles. Unfortunately, the majority of such low-energy electron emitters described in the literature to date have harnessed only a small percentage of the actual cytotoxic potential of auger emitting radionuclides because of poor drug design.

Therefore, there is a need in the art to provide various liposome compositions that are useful for delivery of a variety of compounds, such as, for example, radiotherapeutic, bimodality radiochemotherapeutic, diagnostic, and imaging entities.

### BRIEF SUMMARY OF THE INVENTION

It is now discovered that liposome compositions can be used to overcome the targeting delivery problem of, for example, radiotherapeutics and radiochemotherapeutics. The present invention relates to such liposome compositions that are useful in multifunctional and multimodality radiotherapeutic/radiochemotherapeutic delivery for tumor nuclear imaging and enhanced therapeutic index (e.g., low-energy electron emitters). The delivery of radiotherapeutics and radiochemotherapeutics in accordance with the present invention may be combined with current chemotherapy to provide a more efficient treatment regime.

One aspect of the invention provides a radiolabeled liposome which comprises a liposome composition having a particle forming component and an agent-carrying component enclosed by the particle forming component, and a radiolabeled agent entrapped within the liposome composition, wherein the radiolabeled agent comprises a radionuclide selected from the group consisting of ¹¹¹In, ¹⁷⁷Lu, ⁹⁰Y, ²²⁵Ac, and their daughter radionuclides.

Another aspect of the invention provides a kit for targeting a radiolabeled agent to a tumor site in a subject in need thereof. The kit includes a liposome composition having a particle forming component comprising a vesicle-forming lipid selected from a group of amphipathic lipids having hydrophobic and polar head group moieties alone or in combination, an agent-carrying component enclosed by the particle forming component, wherein the agent-carrying component has a chemical entity that contains one or more negatively charged groups or trapping ions and a radiolabeled agent entrapped within the liposome composition via an electrostatic charge-charge interaction with the agent-carrying component, wherein the radiolabeled agent comprises a radionuclide selected from the group consisting of ¹¹¹In, ¹⁷⁷Lu, ⁹⁰Y, ²²⁵Ac, and their daughter radionuclides. Kits of the invention may further comprise an instruction manual.

A further aspect of the invention provides a method for preparing a radiolabeled liposome, wherein a liposome composition comprising a particle forming component and an agent-carrying component enclosed by the particle forming component is provided. A radiolabeled agent is then entrapped within the liposome composition, wherein the radiolabeled agent comprises a radionuclide selected from the group consisting of ¹¹¹In, ¹⁷⁷Lu, ⁹⁰Y, ²²⁵Ac, and their daughter radionuclides.

Another aspect of the invention provides a method for diagnosing and treating a tumor in a subject in which a liposome composition having a particle forming component, an agent-carrying component and a radiolabeled agent is provided, wherein the agent-carrying component and the radiolabeled agent are enclosed by the particle forming component, and the radiolabeled agent comprises a radionuclide selected from the group consisting of ¹¹¹In, ¹⁷⁷Lu, ⁹⁰Y, ²²⁵Ac, and their daughter radionuclides. The liposome composition is then administered to the subject by, for example, intravenous or intraperitoneal administration.

A further embodiment of the invention provides a nanoparticle for diagnosing and treating a tumor in a subject, wherein the nanoparticle comprises a radionuclide selected from the group consisting of ¹¹¹In, ¹⁷⁷Lu, ⁹⁰Y, ²²⁵Ac, and their daughter radionuclides.

The invention also provides a method for treating a tumor (e.g., a cancer therapy) comprising administering a long-circulating nanoparticle containing a heavy element combined with an antineoplastic agent to a tumor site, wherein the heavy element is selected from the group consisting of ¹¹¹In, ¹⁷⁷Lu, ⁹⁰Y, ²²⁵Ac, and their daughter radionuclides. The tumor site is then irradiated.

Additional aspects and advantages of the invention will be set forth in part in the description which follows, and in part will be apparent from the description, or can be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows a general preparation scheme for indium-111 (In-111 or ¹¹¹In), lutetium-177 (Lu-177 or ¹⁷⁷Lu), yttrium-90 (Y-90 or ⁹⁰Y) or actinium-225 (Ac-225 or ²²⁵Ac) loaded-liposomal vinorelbine (VNB-liposome or NanoVNB);

FIG. 2 shows in vitro labeling stability of 100-nm VNB-liposome labeled with ¹¹¹In-oxine in 95% Human Plasma, pH 7.4, at 37° C.;

FIG. 3A shows the pharmacokinetics of ¹¹¹In-DTPA-liposome (6% PEGDSPE-Liposome-DTPA labeled with ¹¹¹In-oxine), ¹⁷⁷Lu-DTPA-liposome (6% PEGDSPE-Liposome-DTPA labeled with ¹⁷⁷Lu-oxine) and ¹¹¹In-DTPA in normal BALB/c mice;

FIG. 3B shows the pharmacokinetics of ¹¹¹In-VNB-liposome (0.9% PEGDSPE-NanoVNB labeled with ¹¹¹In-oxine) in NOD/SCID mice bearing HT-29 carcinoma;

FIG. 3C illustrates results of the blood clearance test of In-oxine (6% PEGDSPE-NanoVNB labeled with ¹¹¹In-oxine), In-iono-PEG (6% PEGDSPE-NanoVNB labeled with ¹¹¹In-ionophore) and Lu-iono-PEG (6% PEGDSPE-NanoVNB labeled with ¹⁷⁷Lu-ionophore);

FIG. 4A shows gamma scintigraphic images of tumor distribution obtained 48 h postinjection of ¹¹¹In-VNB-liposome;

FIG. 4B shows gamma scintigraphic images of a normal mouse and a HT-29 carcinoma bearing mouse 24 h postinjection of 100 μCi ¹¹¹In-VNB-liposome;

FIG. 4C shows gamma scintigraphic image of a normal mouse and a HT-29 carcinoma bearing mouse 48 h postinjection of 100 μCi ¹¹¹In-VNB-liposome;

FIG. 4D shows gamma scintigraphic images of a normal mouse and a HT-29 carcinoma bearing mouse 24 h postinjection of 100 μCi ¹¹¹In-VNB-liposome;

FIG. 5 shows whole-body autoradiographies (WBARs) of (A) HT-29 carcinoma bearing mice and (B) HT-29/luc carcinoma bearing mice;

FIGS. 6A and 6B show the tumor growth curves in SCID mice inoculated subcutaneously with 2×10⁶ HT-29/luc tumor cells;

FIG. 7 shows the therapeutic efficacy of ¹¹¹In-VNB-liposome in a C26/tk-luc colon carcinoma-bearing mouse model; and

FIG. 8 shows the survival fraction of tumor-bearing mice (n=9) injected intravenously with NanoX (), ¹¹¹In-NanoX (♦), VNB-liposome (▪) or ¹¹¹In-VNB-liposome (▴) at 0, 7, and 14 days.

### DETAILED DESCRIPTION OF THE INVENTION

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

As used herein, “daughter nuclides” are nuclides that are produced in a nuclear decay. While the moment in time at which a particular nucleus decays is unpredictable, a collection of atoms of a radioactive nuclide decays exponentially at a rate described by a parameter known as the half-life, usually given in units of years when discussing dating techniques. After one half-life has elapsed, one half of the atoms of the nuclide in question will have decayed into a “daughter” nuclide (i.e., decay product). In many cases, the daughter nuclide itself is radioactive, resulting in a decay chain, eventually ending with the formation of a stable (i.e., nonradioactive) daughter nuclide. Each step in such a chain is characterized by a distinct half-life. In these cases, the half-life of interest in radiometric dating is usually the longest one in the chain, which is the rate-limiting factor in the ultimate transformation of the radioactive nuclide into its stable daughter.

As used herein, “heavy elements” refers to a group of elements that exhibit metallic properties, including but not limited to, the transition metals of the periodic table, some metalloids, lanthanides, and actinides, and their daughter radionuclides.

As used herein, “NanoVNB” or “VNB-liposome” is a liposome composition comprising Vinorelbine (VNB) encapsulated in a NanoX liposome. NanoX is a vehicle for drug loading, comprising small unilamellar liposomes, e.g., having a mean diameter of approximately 100 nm. Vinorelbine is an anti-mitotic chemotherapy drug that is used as a treatment for some types of cancer, including but not limited to breast cancer and non-small cell lung cancer. Other antineoplastic agents or chemotherapeutic agents can also be encapsulated together with a radioactive agent in accordance with the present invention. Exemplary antineoplastic agents include but are not limited to a vinca derivative drug, vinorelbine, vincristine, vinblastine, vinflunine; an anthracycline drug, doxorubicin, daunorubicin, mitomycin C, epirubicin, pirarubicin, rubidomycin, carcinomycin, N-acetyladriamycin, rubidazone, 5-imidodaunomycin, N-acetyldaunomycine, daunoryline, mitoxanthrone, a camptothecin compound, camptothecin, 9-aminocamptothecin, 7-ethylcamptothecin, 10-hydroxycamptothecin, 9-nitrocamptothecin, 10,11-methylenedioxycamptothecin, 9-amino-10,11-methylenedioxycamptothecin, 9-chloro-10,11-methylenedioxycamptothecin, irinotecan, topotecan, lurtotecan, silatecan, (7-(4-methylpiperazinomethylene)-10,11-ethylenedioxy-20(S)-camptothecin, 7-(4-methylpiperazinomethylene)-10,11-methylenedioxy-20(S)-camptothecin, 7-(2-N-isopropylamino)ethyl)-(20S)-camptothecin, an ellipticine compound, ellipticine, 6-3-aminopropyl-ellipticine, 2-diethylaminoethyl-ellipticinium and salts thereof, datelliptium, and retelliptine.

The term “neovascularization” as used herein refers to abnormal growth of blood vessels, for example, at or near an area of a tumor.

The present invention provides a liposome composition for delivering high pay-load of a radiotherapeutic or radiochemotherapeutic agent to neovascularization sites of a tumor or a cancer in a patient in need thereof. According to embodiments of the invention, the liposome composition is a submicro-sized or nano-sized particle that comprises a particle-forming component and an agent-carrying component. The submicro-size particles have a mean particle diameter of about 100 nm to about 400 nm, more particularly about 100 nm to about 200 nm. The nano-sized particles has a mean particle diameter of about 30 nm to about 100 nm, more particularly about 50 nm to about 100 nm. The particle-forming component forms an enclosed lipid barrier of the particle. The agent-carrying component interacts with an encapsulated agent, such as a radiotherapeutic or radiochemotherapeutic agent, by electrostatic charge-charge interaction to form a stable complex or to remove a carrier for the encapsulated agent, such as oxine or ionophore, to enhance the hydrophilicity, thus to stabilize the encapsulated agent inside the vesicle. The hydrophilicity of an encapsulated agent, such as a radiotherapeutic or radiochemotherapeutic agent, prevents or minimizes the release of the agent from the liposome particle in blood circulation and allows high pay-load of the agent to be delivered to target tissues, including neovascularization sites of the tumor.

According to an embodiment of the invention, the liposome composition comprising the radiotherapeutic or radiochemotherapeutic agent is systemically administered to the subject. In a particular embodiment of the invention, the liposome composition comprising the radiotherapeutic or radiochemotherapeutic agent is intravenously or intraperitoneally administered to the subject, and the therapeutic agent entrapped in the liposome composition is accumulated at a neovascularization site of a tumor after the administration (e.g., at about 24 hours after administration). The subjects to which administration of the liposome compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

In accordance with another embodiment of the invention, a kit is provided for targeting a radiolabeled agent to a tumor site in a subject in need thereof. The kit includes a liposome composition comprising a particle forming component comprising a vesicle-forming lipid selected from a group of amphipathic lipids having hydrophobic and polar head group moieties alone or in combination, an agent-carrying component enclosed by the particle forming component, wherein the agent-carrying component has a chemical entity that contains one or more negatively charged groups or trapping ions and a radiolabeled agent entrapped within the liposome composition via an electrostatic charge-charge interaction with the agent-carrying component, wherein the radiolabeled agent comprises a radionuclide selected from the group consisting of ¹¹¹In, ¹⁷⁷Lu, ⁹⁰Y, ²²⁵Ac, and their daughter radionuclides, and an instruction manual.

In a further embodiment of the invention, a method for preparing a radiolabeled liposome is provided. The method includes providing a liposome composition comprising a particle forming component and an agent-carrying component enclosed by the particle forming component. A radiolabeled agent is then entrapped within the liposome composition, wherein the radiolabeled agent comprises a radionuclide selected from the group consisting of ¹¹¹In, ¹⁷⁷Lu, ⁹⁰Y, ²²⁵Ac, and their daughter radionuclides.

Another embodiment of the invention provides a method for diagnosing and treating a tumor in a subject. The method comprises providing a liposome composition having a particle forming component, an agent-carrying component and a radiolabeled agent, wherein the agent-carrying component and the radiolabeled agent are enclosed by the particle forming component, and the radiolabeled agent comprises a radionuclide selected from the group consisting of ¹¹¹In, ¹⁷⁷Lu, ⁹⁰Y, ²²⁵Ac, and their daughter radionuclides. The liposome composition is then administered to the subject by, for example, intravenous or intraperitoneal administration. In other embodiments, the radionuclide may also be entrapped within another carrier such as a nanoparticle that provides a means for diagnosing and treating a tumor in a subject.

In addition, the present invention provides a method for treating a tumor in a subject (e.g., a cancer therapy) comprising administering to the subject a long-circulating nanoparticle containing a heavy element combined with an antineoplastic agent to a tumor site, wherein the heavy element is selected from the group consisting of ¹¹¹In, ¹⁷⁷Lu, ⁹⁰Y, ²²⁵Ac, and their daughter radionuclides in order to position the heavy element and antineoplastic agent in close proximity to the endothelial cells of blood vessels of neovasculating areas of the tumor. The tumor site is then irradiated so as to cause concurrent chemoradiotherapy.

A detailed description of exemplary particle-forming components and agent-carrying components for preparing the liposome compositions of the invention are set forth below.

Particle-Forming Component

In one embodiment of the invention, the particle-forming component for use in the present invention comprises a variety of vesicle-forming lipids, including, but not limited to, any amphipathic lipids having hydrophobic and polar head group moieties, such as phospholipids, diglycerides, dialiphatic glycolipids, sphingomyelin, glycosphingolipid, cholesterol and derivatives thereof, alone or combinations thereof.

Particular vesicle-forming lipids for use in embodiments of the present invention are those having two hydrocarbon chains, typically acyl chains, and a polar head group. Phospholipids, such as phosphatidic acid (PA), phosphatidylcholine (PC), phosphatidylglycerol (PG), phosphatidylethanolamine (PE), phosphatidylinositol (PI), phosphatidylserine (PS) and sphingomyelin (SM), each having two hydrocarbon chains ranging from about 12-22 carbon atoms in length, and with varying degree of unsaturation, can be used as the particle-forming component according to embodiments of the present invention. In particular aspects of the invention, the vesicle-forming lipid is a phospholipid having a long carbon chain of (—CH₂)ₙ, wherein n is at least 14. These phospholipids may be naturally occurring or synthetic. Naturally occurring phospholipids may also be modified by subjecting to various degrees of hydrogenation.

The particle-forming component may contain a hydrophilic polymer that has a long chain of a highly hydrated flexible neutral polymer attached to a lipid molecule. Examples of the hydrophilic polymer include, but are not limited to, polyethylene glycol (PEG), polyethylene glycol derivatized with Tween, polyethylene glycol derivatized with distearoylphosphatidylethanolamine (PEG-DSPE), ganglioside GM₁, and synthetic polymers. In one embodiment of the invention, the hydrophilic polymer is PEG having a molecular weight of about 500 to about 5,000 daltons. In one particular embodiment, PEG has a molecular weight of approximately 2,000 daltons. It has been reported that PEG-PE incorporation in liposomes produces steric stabilization resulting in longer circulation times in blood (Lasic et al., Biochim. Biophys. Acta, 1070: 187-192, 1991; Papahadjopoulos et al., Proc. Natl. Acad. Sci. U.S.A, 88: 11460-11464, 1991; Gabizon et. al., Biochim. Biophys. Acta, 1103: 94-100, 1992).

In addition, the particle-forming component may further comprise a lipid-conjugate of an antibody or a peptide that acts as a targeting moiety to enable the submicro-sized or nano-sized particle to specifically bind to a target cell bearing a target molecule (e.g., a cell surface marker to which the antibody or peptide is directed). Cell surface markers include, but are not limited to, epidermal growth factor receptor (EGFR), vascular endothelial growth factor receptor (VEGFR), and erbB-2/neu (Her2) (Park et al., Clin. Cancer Res., 8: 1172-1181, 2002; Park et al., J. Control Release, 74: 95-113, 2001; Park et al., Adv. Pharmacol., 40: 399-435, 1997; Mamot et al., Cancer Res., 63: 3154-3161, 2003).

The particle-forming component may also include a lipid-conjugate of an antibody or a peptide that acts as a targeting moiety to enable the submicro-sized or nano-sized particle to specifically bind a target disease site bearing a target molecule (e.g., a disease-specific marker to which the antibody or peptide is directed). Disease-specific markers include, but are not limited to vascular endothelial growth factor/receptor (VEGF/VEGFR) and carcinoembryonic antigen (CEA).

Agent-Carrying Component

As described herein above, the agent-carrying component has the ability to form a complex with an encapsulated agent (e.g., a radiotherapeutic or radiochemotherapeutic agent) via an electrostatic charge-charge interaction. The agent-carrying complex may also have the ability to remove a carrier for the encapsulated agent, such as oxine or ionophore, to enhance the hydrophilicity, thereby stabilizing the encapsulated agent, inside the vesicle. The agent-carrying component can be any suitable chemical entity that contains one or more negatively or positively charged groups. The chemical entity may be charged by deprotonation to form a negatively charged agent-carrying component or by protonation to form a positively charged agent-carrying component.

A negatively charged agent-carrying component according to embodiments of the present invention may be, for example, a divalent anion, a trivalent anion, a polyvalent anion, a polymeric polyvalent anion, a polyanionized polyol, or a polyanionized sugar. Examples of divalent and trivalent anions include, but are not limited to, sulfate, phosphate, pyrophosphate, tartrate, succinate, maleate, borate, and citrate. Polyanionic polymers have an organic or inorganic backbone and a plurality of anionic functional groups. Examples of polyanionic polymers include, but are not limited to, polyphosphate, polyvinylsulfate, polyvinylsulfonate, polycarbonate, acidic polyaminoacids, and polynucleotides.

A positively charged agent-carrying component according to embodiments of the present invention can be any organic polycationics such as polyamines, polyammonium molecules, and basic polyamino acids. In addition, the agent-carrying component can be a chelating agent that forms a chelating complex with a divalent or trivalent cation, including, for example, a transition metal such as lutetium, yttrium, actinium, indium, nickel, iron, cobalt, calcium, magnesium ions. Examples of chelating agents include, but are not limited to ethylenediaminetetraacetic acid (EDTA), diethylenetriaminepentaacetic acid (DTPA), 1,4,7,10-tetraazacyclododecane-1,4,7,10-tetraacetic acid (DOTA), nitroltriacetic acid (NTA), deferoxamine, and dexrozpxane.

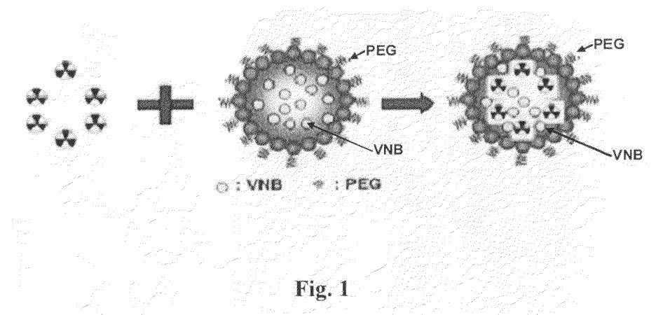

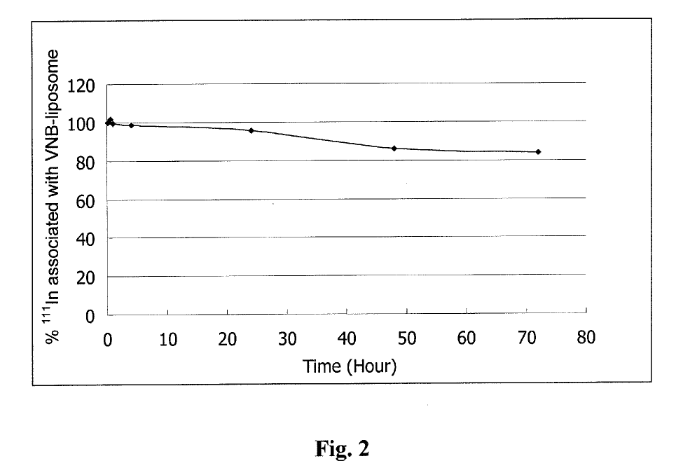

The liposome compositions according to particular embodiments of the present invention comprise a radiotherapeutic or radiochemotherapeutic agent and an agent-carrying component entrapped in a particle-forming component, as illustrated in FIG. 1 and Tables 1A and 1B. Such liposome compositions stably encapsulate the radiotherapeutic or radiochemotherapeutic agent so that little radiotherapeutic or radiochemotherapeutic agent is separated from the particle-forming component after an extended period of time in blood plasma at physiological conditions. As shown in FIG. 2, less than 20% of the radiochemotherapeutic agent is separated from the particle-forming component after a 72 hour incubation in blood plasma at 37° C.

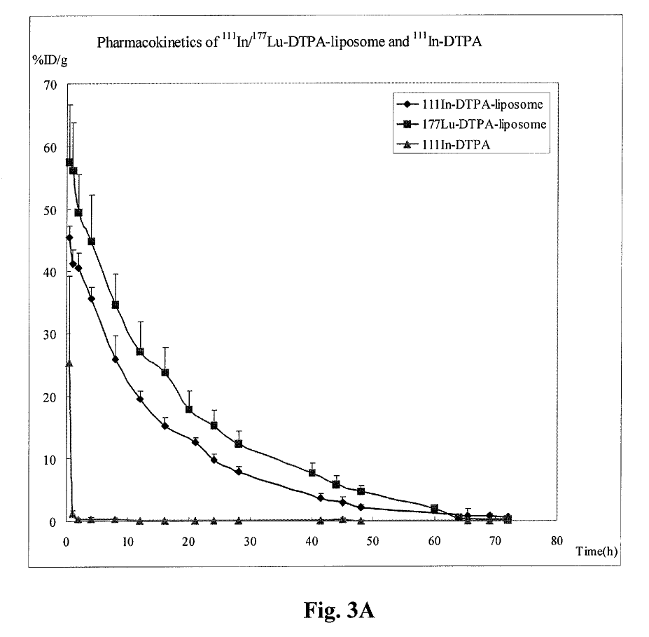

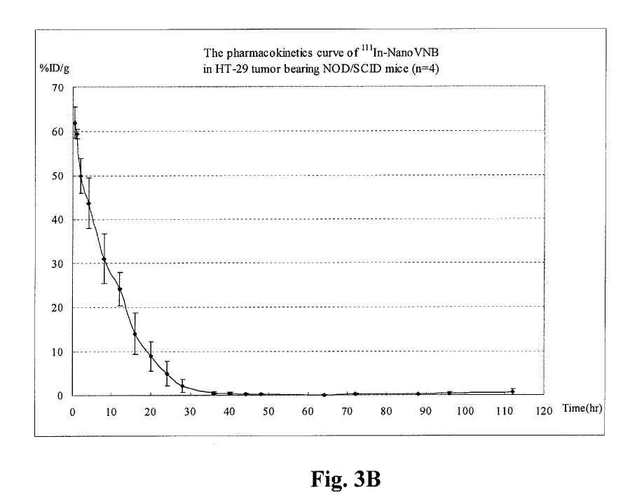

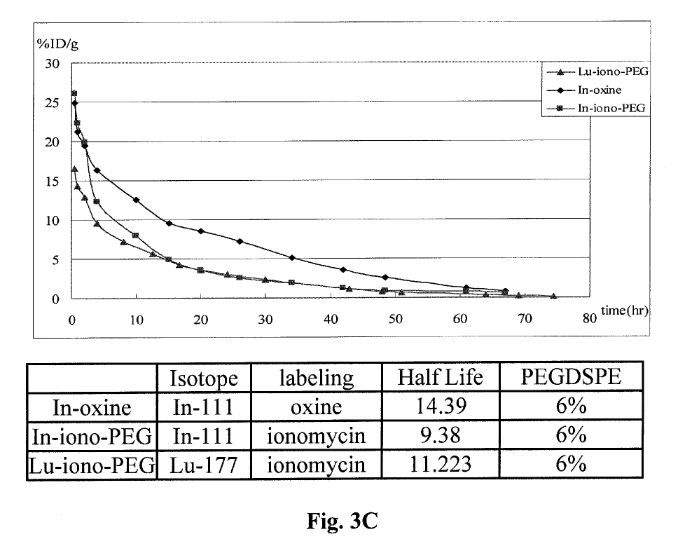

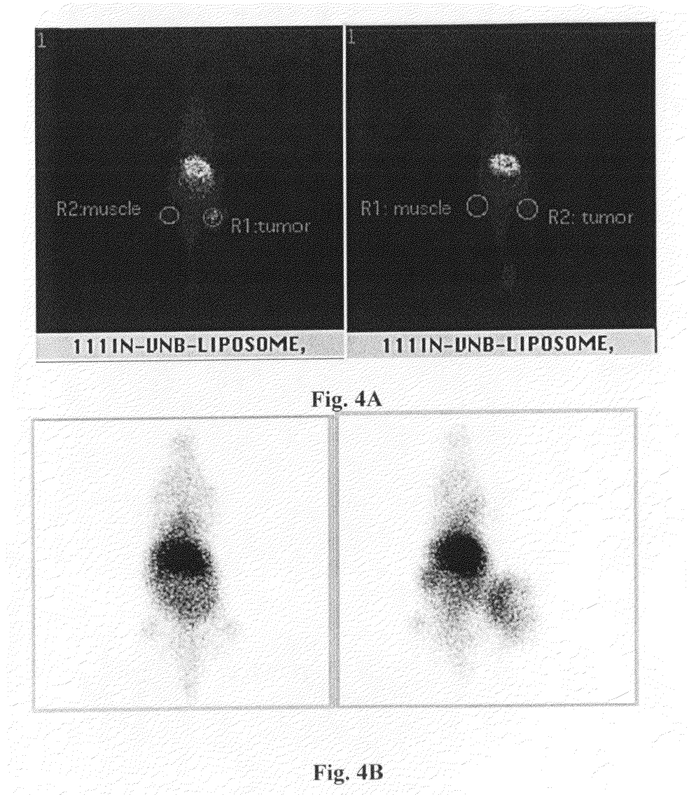

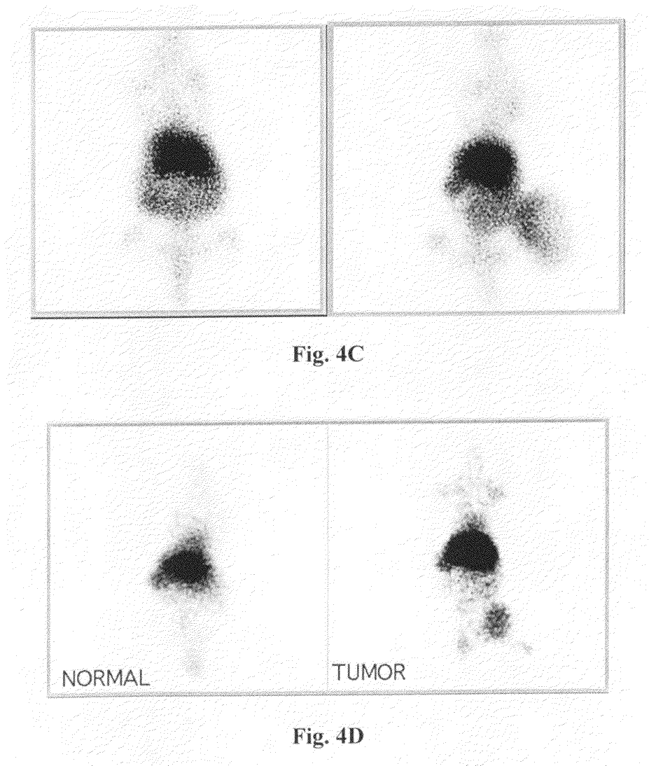

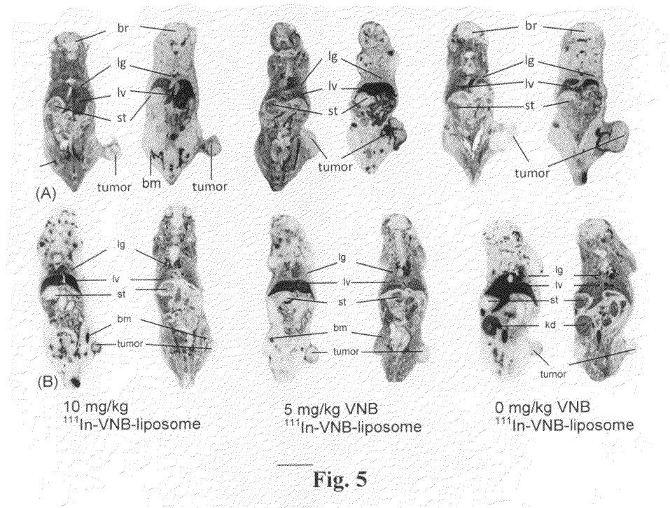

Liposome compositions according to other embodiments of the present invention are long-circulating, as shown in FIGS. 3A through to 3C, and systemically deliver high pay-load of a therapeutic agent to neovascularization sites (e.g., pathological neovascularization sites associated with, for example, a tumor as shown. See FIGS. 4A through to 4D and FIG. 5. It is understood by one having ordinary skill in the art that delivery of a high pay-load of a therapeutic agent to neovascularization sites associated with other diseases, such as inflammation, may also be achieved by the liposome system according to embodiments of the present invention.

The following examples illustrate a method of delivering a high pay-load of a radiotherapeutic or radiochemotherapeutic agent via the blood stream to a neovascularization site of a tumor, thereby enhancing the therapeutic efficacy of auger electron emitting radionuclides. The examples are in no way intended to limit the scope of the present invention.

Radiochemical Synthesis of ¹¹¹In-oxine and ¹⁷⁷Lu-oxine

A total of 300 μL of 0.69 or 69 mM 8-hydroxyquinoline (oxine, Sigma-Aldrich Co., St. Louis, Mo., USA) in ethanol was added to 300 mL of ¹¹¹InCl₃ (indium chloride, Perkin Elmer, Boston, Mass.) in 0.05M sodium acetate buffer (pH 6-7). The mixture was then incubated at 50° C. for 30 min. The lipophilic components in the mixture were extracted with methylene chloride. The organic layer was then dried with anhydrous sodium sulfate. The labeling efficiency of ¹¹¹In-oxine was determined by instant thin layer chromatography (ITLC).

¹⁷⁷Lu-oxine was prepared and analyzed following the same procedure as that for ¹¹¹In-oxine described above. The radiochemical yield was generally greater than 90% for ¹¹¹In-oxine and about 70% for ¹⁷⁷Lu-oxine.

Alternatively, ¹¹¹In-oxine was synthesized according to the following procedure. 100 μg of 8-hydroxyquinoline (oxine; Sigma-Aldrich Co., St. Louis, Mo.) in 10 μl of ethanol was added to 40 μl of ¹¹¹InCl₃ (indium chloride in 0.05M HCl, Perkin Elmer, Boston, Mass.) in 0.2 M sodium acetate buffer (pH5.5). The mixture was incubated at 50° C. for about 15 min. The labeling efficiency of ¹¹¹In-oxine was analyzed by instant thin layer chromatography (ITLC). The lipophilic product in the mixture was extracted by one milliliter of chloroform. The extraction efficiency was determined by measuring the radioactivity in the buffer and the chloroform phase. The extracted ¹¹¹In-oxine in the chloroform phase was evaporated to dryness at 60° C. for one hour. The lipophilic product was dissolved in 10 μl of ethanol, followed by the addition of 40 μl of water.

Preparation of Liposomes (Nanox)

Small unilamellar vesicles (size ˜100 nm) were prepared by a combination of the standard thin-film hydration method and repeated extrusion. Distearoylphosphatidylcholine (DSPC), Cholesterol and 0.9 mol % PEG-DSPE (molar ratio, 3:2:0.045) or DSPC, Cholesterol and 6 mol % PEG-DSPE (molar ratio, 3:2:0.3) were dissolved in chloroform and placed in a round-bottomed flask. The solvent was removed by rotary evaporation under reduced pressure. The resulting dry lipid film was hydrated at 60° C. in aqueous solution (TEA-SOS, 0.6 M triethylammonium, pH 5.7-6.2) and dispersed by hand shaking at 60° C. The suspension was frozen and thawed five times followed by repeated extrusion through polycarbonate membrane filters (Costar, Cambridge, Mass., USA) of 0.1 μm pore size (three times) and 0.05 μm pore size (seven times) by using high-pressure extrusion equipment (Lipex Biomembranes, Vancouver, BC) at 60° C. After extrusion, the extraliposomal salt was removed by using a Sephadex G-50 column eluted with histidine-sucrose buffer (24 mM histidine hydrogen chloride, 90 μL sucrose, pH adjusted to 6.0 with NaOH).

Anticancer Drug Encapsulation

The anticancer agent VNB was encapsulated into the nanoliposomes (100 nm in diameter) using a polyanionic gradient. After the extraliposomal salt was removed by using the Sephadex G-50 column, VNB was added immediately into the solution at a concentration of 3.5 mg VNB per 10 μmol phospholipid. The mixture of liposomes and VNB was incubated in a 60° C. water bath for 30 min with agitation (100 rpm). After loading, the liposomal VNB was sterilized by 0.2 μm filtration and stored at 4 to 6° C. before use. The liposomes (NanoVNB) were characterized by lipid concentration, drug concentration and particle size: pH=6.1, osmolarity=361 mmol/kg, mean particle size=95.2 nm, phospholipids=6.19 mol/ml, VNB=2.08 mg/ml.

### Preparation of DTPA Encapsulated Liposomes (Lipo-DTPA)

Small unilamellar vesicles (size ˜100 nm) were prepared by a combination of the standard thin-film hydration method and repeated extrusion. DSPC, Cholesterol and 0.9 mol % PEG-DSPE (molar ratio, 3:2:0.045) or DSPC, Cholesterol and 6 mol % PEG-DSPE (molar ratio, 3:2:0.3) were dissolved in chloroform and placed in a round-bottomed flask. The solvent was removed by rotary evaporation under reduced pressure. The resulting dry lipid film was hydrated at 60° C. in aqueous solution (DTPA (diethylenetriaminepentaacetate, Sigma-Aldrich Co., St. Louis, Mo.), 10 mM triethylammonium, 144 mM NaCl, pH 7.2) and dispersed by hand shaking at 60° C. The suspension was frozen and thawed five times followed by repeated extrusion through polycarbonate membrane filters (Costar, Cambridge, Mass., USA) of 0.1 μm pore size (three times) and 0.05 μm pore size (seven times) by using high-pressure extrusion equipment (Lipex Biomembranes, Vancouver, BC) at 60° C. After extrusion, the extraliposomal salt was removed by using the Sephadex G-50 column eluted with normal saline.

Liposome Labeling with ¹¹¹In-oxine or ¹⁷⁷Lu-oxine

Liposomes labeling was performed by incubating 2 mCi of ¹¹¹In-oxine or ¹⁷⁷Lu-oxine with 2 ml of NanoX (TEA-SOS encapsulated liposome), liposomal vinorelbine (NanoVNB, Taiwan Liposome Co., Ltd. Taipei, Taiwan) or DTPA (Sigma-Aldrich Co., St. Louis, Mo.) encapsulated liposome (Lipo-DTPA) for 30 minutes at room temperature. The labeled liposomes were assayed by loading 20 μl of sample onto a 2 ml Sephadex G50 column (Pharmacia, Uppsala, Sweden). Thirty consecutive 0.2 ml fractions were eluted with normal saline and the radioactivity of each fraction was counted in a gamma counter. Indium-111 or lutetium-177 entrapment was greater than 90%.

Cancer Cell Lines and Culture Condition

The HT-29 colonrectal carcinoma cell line was obtained from the Taipei Veterans General Hospital. The murine colon adenocarcinoma cell line, C26, which was originally induced by N-nitroso-N-methylurethan (NNMU) in BALB/c mouse, was generously provided by Taiwan Liposome Co., Ltd. (Taipei, Taiwan). The cell lines were cultured in RPMI-1640 with 10% fetal bovine serum (Hyclone) and supplemented with L-glutamine, sodium pyruvate, non-essential amino acids (Hyclone). The cell lines were maintained at 37±2° C. in a humidified atmosphere containing 5% CO₂.

Transfection

Transfection of the HT-29 cell line with luc, the bioluminescence gene, was performed using jetPEI (polyplus-transfection). The transfected cells were selected with 500 μg/ml G418 (Merck). The surviving colonies were screened for bioluminescence by in vitro bioluminescence imaging (BLI) using the IVIS 50 Imaging System as described below (Xenogen Corporation, Alameda, Calif.). The transfected clone, HT-29/luc, was characterized by stable luminescence expression in vitro and tumorigenic potential in vivo.

Transfection of the C26 cell line with tk-luc, the bioluminescence gene, was performed using jetPEI (polyplus-transfection). The transfected cells were selected with 500 μg/ml G418 (Merck). The surviving colonies were screened for bioluminescence by in vitro bioluminescence imaging (BLI) using the IVIS 50 Imaging System as described below (Xenogen Corporation, Alameda, Calif.). The transfected clone, C26/tk-luc, was characterized by stable luminescence expression in vitro and tumorigenic potential in vivo.

C26 Animal Tumor Model

Five- to six-week-old male BALB/c mice were purchased from National Taiwan University. All animal experiments were performed in accordance with the approved protocols and recommendations for the proper care and use of laboratory animals. To establish the malignant solid tumor, 2×10⁵ of C26 cells were inoculated into BALB/c mice subcutaneously on day 0. The volume of injection was 100 μl per mouse. This volume prevented leakage and maintained the integrity of the cell contents during implantation into the mice.

HT-29/luc Animal Tumor Model (HT-29/luc Mice)

Human colorectal carcinoma (HT-29/luc) tumor models was established in SCID mice. SCID mice were anesthetized by intramuscular injection of 100 mg/kg ketamine hydrochloride plus 6 mg/kg xylazine. Each of the mice received about 2×10⁶ or 10⁷ HT-29/luc cells in a subcutaneous injection below the dorsal flank. The injected cells were suspended in RPMI-1640 medium to about 100 μl. Tumor volume caliper measurements (L×W×D×0.523) began 10 days after injection.

The Plasma Stability of ¹¹¹In-Liposome

About 0.2 ml of a labeled liposome preparation (approximate 0.1 to 0.3 mCi) was added to about 3.8 ml human plasma (obtained from Taipei Veterans General Hospital). The mixture was incubated at 37° C. water bath immediately. At time points of 0.083 (5 minutes), 0.5, 1, 4, 24, 48 and 72 hours after the incubation, 200 μl aliquot samples of the mixture were withdrawn. Free In-111 was separated from ¹¹¹In-liposome in each aliquot sample by Sepharose™ CL-4B gel filtration by following steps. About 2-ml Sephadex™ CL-4B gel was packed into a Poly-Prep Column, and washed with 10 ml 9% NaCl solution (normal saline). The 200 μl aliquot sample was applied to the Sepharose CL-4B gel packed column, followed by washing the gel with 450 μl of normal saline. The gel was further washed with 600 μl of normal saline. The elution solution was collected in 1.5 ml eppendorf tubes. The amounts of ¹¹¹In and phospholipids in the pre-separation aliquot sample and in the elution solution were measured. The encapsulation ratio was determined by comparing the amount of encapsulated ¹¹¹In with the amount of phospholipids in each sample. The plasma stability of ¹¹¹In-Liposome, such as ¹¹¹In-NanoVNB liposome, was determined by comparing the encapsulation ratio before and after in vitro incubation in human plasma.

The Pharmacokinetics Study of ¹¹¹In-Liposome

Eight NOD/SCID mice bearing HT-29 carcinoma (HT-29 mice) were separated equally into 2 groups. Mice in each group were injected intravenously with ¹¹¹In-Lipo-DTPA and ¹¹¹In-NanoVNB, respectively. The injected radioactivity was about 40 to 50 μCi. Blood samples from the mice were collected from the tail vein of the mice at 0.5, 1, 2, 4 hours till 96 or 112 hours postinjection. Radioactivity in each of the blood samples was measured by a γ-counter and output data were analyzed.

To investigate the blood clearance rate of ¹¹¹In-Lipo-DTPA and ¹⁷⁷Lu-Lipo-DTPA, 40 μCi each of ¹¹¹In-Lipo-DTPA, ¹⁷⁷Lu-Lipo-DTPA and ¹¹¹In-DTPA was injected into four NOD/SCID mice via tail vein injection.

To investigate the blood clearance rate of ¹¹¹In-NanoVNB, 50 μCi ¹¹¹In-NanoVNB was injected into four NOD/SCID mice via tail vein injection.

Biodistribution Study of ¹¹¹In Labeled Radiopharmaceuticals In Vivo

In vivo biodistribution analyses were performed with tumor bearing mice on the day when tumor volume approached 500-600 mm³. HT-29 tumor bearing NOD/SCID mice were injected intravenously with about 100 μCi ¹¹¹In labeled radiopharmaceuticals. At 1, 4, 24, 48, and 72 hours after drug injection, mice were sacrificed. Tumors, heart, blood, lung, liver, pancreas, kidneys, stomach, small intestine, large intestine, spleen, muscle and bone were removed for radioactivity measurement with a gamma scintillation counter. The uptake of ¹¹¹In labeled radiopharmaceuticals in the tumor and tissues are expressed in counts per minutes and are normalized as percentage injection dose (% ID) per gram tissue:

%ID/g=A₀×1000/(ID(μCi)×3.7×10⁴×60×Eff×organ weight (mg),

wherein ln (A/A₀) 0.693t/t_{1/2}, A=radioactivity (cpm) of tissues or organs measured by γ-counter, A₀=decay-corrected radioactivity (cpm) of tissues or organs, Eff=counting efficiency of γ-scintillation counter, t_{1/2}=half-life of radioisotope, and t=time after injection.

HT-29 and HT-29/luc Tumor-Bearing Animal Model and ¹¹¹In-NanoVNB Administration

Male NOD/SCID mice (purchased from Tzu Chi University, Hwalien, Taiwan) were i.p. anesthetized with ketamine hydrochloride plus xylazine. About 2×10⁶ HT-29 or HT-29/luc cells were implanted subcutaneously at the dorsal flanks of mice. Perpendicular tumor diameters were measured 10 days after injection or until the bulge was observed using a Vernier scale caliper. Tumor volume was estimated by the formula: ½× 4/3π×length/2×width/2×thickness=0.523×(length×width×thickness). Treatment with a liposome composition or a control was initiated when tumor volume was about 100 mm³. HT-29 mice or HT-29/luc mice were divided into 3 experimental groups, subject to the treatment of 5 mg/kg ¹¹¹In-NanoVNB (radiochemotherapy), ¹¹¹In-NanoX (radiotherapy), and normal saline (control), respectively. The ¹¹¹In-NanoVNB was administered once a week for 4 weeks with a maximum accumulation dose of 20 mg/kg VNB and 400 μCi ¹¹¹In encapsulated in ¹¹¹In-NanoVNB. The ¹¹¹In-NanoX was also administered once a week for 4 weeks with a maximum accumulation dose of 400 μCi ¹¹¹In encapsulated in ¹¹¹In-NanoX.

Therapeutic Efficacy of ¹¹¹In-VNB-Liposome in a C26/tk-luc Colon Carcinoma-Bearing Mouse Model

Five- to six-week-old male BALB/c mice were purchased from National Taiwan University. All animal experiments were performed in accordance with the approved protocols and recommendations for the proper care and use of laboratory animals. To establish the malignant solid tumor, 2×10⁵ of C26/tk-luc cells were inoculated into BALB/c mice subcutaneously on day 0. The volume of injection was 100 μl per mouse. This volume prevented leakage and maintained the integrity of the cell contents during implantation into the mice. Perpendicular tumor diameters were measured 10 days after injection or until the bulge was observed using a Vernier scale caliper. Tumor volume was estimated by the formula: ½× 4/3π×length/2×width/2×thickness=0.523×(length×width×thickness). Treatment with a liposome composition or a control was initiated when tumor volume was about 75 mm³. C26/tk-luc mice were divided into 4 experimental groups (n=9 for each group), subject to the treatment intravenously of ¹¹¹In-NanoVNB (3 mCi and 3 mg/kg vinorelbine, radiochemotherapy), ¹¹¹In-NanoX (3 mCi, radiotherapy), NanoVNB (3 mg/kg vinorelbine, chemotherapy) and NanoX (Control) at 0, 7, and 14 days.

Radiochemical Synthesis of ¹¹¹In-oxine and ¹⁷⁷Lu-oxine

The radiochemical purity of ¹¹¹In-oxine was up to 95.20±3.90% and the radiolabeling efficiency for ¹¹¹In-oxine was more than 90%. As shown in Table 1A, the concentration of oxine used in the labeling reaction affected the radiolabeling efficiency, particularly that of ¹⁷⁷Lu-oxine. At a concentration of 6.9 mM (0.1 mg/ml), the radiolabeling efficiency was good for 111In, e.g., more than 90%, but poor for ¹⁷⁷Lu, e.g., ≦10%). Increasing the oxine concentration to 34.5 mM elevated the radiolabeling efficiency for ¹⁷⁷Lu to 70-80%. Higher oxine concentration, e.g., 69 mM, increased radiolabeling efficiency for ¹⁷⁷Lu further (data not shown). Temperature and incubation time also affected the radiolabeling efficiency. For 0.69 mM oxine solution, the optimized temperature of incubation was 50° C. and the radiolabeling efficiency was further improved by longer incubation time (data not shown). For 69 mM oxine solution, however, there was no beneficial effect with incubation time exceeding 30 minutes (data not shown).

TABLE 1A

| | | | | | Labeling |
|---|---|---|---|---|---|
| | Oxine concentration | | pH | Temp. and Time | efficiency |
| ¹¹¹In-oxine | Low | (0.69 mM) | 5.5 | 50° C.; 30 mins | >90% |
| | Low | (0.69 mM) | 6-7 | 50° C.; 30 mins | >90% |
| | High | (69 mM) | 6-7 | 50° C.; 30 mins | >90% |
| ¹⁷⁷Lu-oxine | Low | (0.69 mM) | 4-5 | 50° C.; 30 mins | <5% |
| | Low | (0.69 mM) | 6-7 | 50° C.; 30 mins | 5~10% |
| | High | (34.5 mM) | 6-7 | 50° C.; 30 mins | 70~80% |

In a preferred embodiment, the radiolabeling conditions comprise a temperature of 50° C. and an incubation time of 30 minutes. Under these conditions, a radiolabeling efficiency of 89.23±1.12% (n=5) was reached for ¹¹¹In-oxine and about 70% for ¹¹¹Lu-oxine.

Liposome Labeling with ¹¹¹In-oxine

Metal chelator, DTPA, polysulfate or sucrose octasulfate, encapsulated liposomes can load indium or lutetium into the interior of liposome as illustrated in FIG. 1. Sephadex™ G-50 packed fine column was used for loading efficiency analysis. Sephadex™ G-50 fine column was washed with 10 ml normal saline first. Then, about 100 μL labeled liposome composition was applied to Sephadex™ G-50 fine column followed by washing with normal saline. The labeling efficiency of ¹¹¹In-oxine with NanoVNB (sucrose octasulfate and vinorelbine encapsulated liposome) was given below. As shown in Table 1B, the loading efficiency of ¹¹¹In to NanoVNB liposome particles was more than 90% when the loading ratio of ¹¹¹In to the liposome particles was at about 1 or less. Also, Table 1C below shows the loading efficiency of ¹⁷⁷Lu-oxine to liposomes.

TABLE 1B

| | | | Loading ratio | | |
|---|---|---|---|---|---|
| | | Liposome content | (¹¹¹In/liposome | Temper- | Loading |
| ¹¹¹In-oxine | | (phospholipids) | particle) | ature | efficiency |
| 70 | μCi | 492.75 nmole | 0.12 | 37° C. | 95% |
| 100 | μCi | 492.75 nmole | 0.17 | 37° C. | 94.3% |
| 200 | μCi | 492.75 nmole | 0.34 | 37° C. | 93% |
| 300 | μCi | 492.75 nmole | 0.52 | 37° C. | 94% |
| 600 | μCi | 492.75 nmole | 1.04 | 37° C. | 91% |
| 1200 | μCi | 492.75 nmole | 2.08 | 37° C. | 84% |
| 2400 | μCi | 492.75 nmole | 4.16 | 37° C. | 71.6% |
| 4800 | μCi | 492.75 nmole | 8.32 | 37° C. | 73% |
| 9600 | μCi | 492.75 nmole | 16.64 | 37° C. | 71.4% |

TABLE 1C

| | Loading ratio | | | |
|---|---|---|---|---|
| Liposome | (¹⁷⁷Lu/liposome | | Incubation | Loading |
| formulation | particle | Temperature | time | efficiency |
| NanoVNB | 0.10 | 37° C. | 1 hour | 68.7% |
| NanoX | 0.09 | 37° C. | 1 hour | 70.6% |
| Lipo-DTPA | 0.11 | 37° C. | 1 hour | 67.7% |
| Lipo-DTPA | 0.10 | 37° C. | 0.5 hour | 54.5% |

Stability of Labeled Liposome in Plasma

As shown in FIG. 2, ¹¹¹In-NanoVNB was stable in human plasma for a relatively long period of time. About 95.63% of ¹¹¹In remained to be encapsulated after about 24 hours incubation. The stability gradually decreased to about 85.76% after about 48 hours incubation. There was only about 2 percent (85.76% to 83.91%) decrease of encapsulated ¹¹¹In during further incubation from 48 to 72 hours, suggesting that the liposome composition was relatively stable during this period of incubation.

The stability of drug-loaded liposomes over time is an important concern in pharmaceutical formulations. In vitro stability studies using human plasma often correlate to the pharmacokinetic property in vivo.

Pharmacokinetics of ¹¹¹In-Lipo-DTPA, ¹⁷⁷Lu-Lipo-DTPA and ¹¹¹In-NanoVNB

FIG. 3A illustrates the plasma concentration-time profile of 40 μCi each of ¹¹¹In-Lipo-DTPA (also named ¹¹¹In-DTPA-Liposomse), ¹⁷⁷Lu-Lipo-DTPA (also named ¹⁷⁷Lu-DTPA-Liposomse), and ¹¹¹In-DTPA (a non-liposome control) from an in vivo blood clearance study in normal BALB/c mice. The semi-logarithmic plot of plasma concentration versus time appears to indicate that ¹¹¹In-Lipo-DTPA and ¹⁷⁷Lu-Lipo-DTPA were eliminated from a single compartment by a first order process with a half-life of about 10.2 and about 11.5 hours.

FIG. 3B illustrates the plasma concentration-time profile for 50 μCi of ¹¹¹In-NanoVNB from an in vivo blood clearance study in NOD/SCID mice bearing HT-29 carcinoma. The semi-logarithmic plot of plasma concentration versus time showed more complicated patterns than mono exponential kinetics. Before 36 hours, it appears that the ¹¹¹In-NanoVNB was eliminated by a first order process, which suggested that pharmacokinetic characteristic was one-compartment model during this time period, with a half-life of 7.09 hours (γ²=0.9945). However, after 36 hours, no significant elimination of ¹¹¹In-NanoVNB and stable plasma concentration were observed. As shown in FIG. 3A, ¹¹¹In-DTPA-liposome and ¹⁷⁷Lu-DTPA-liposome, as steric stabilized liposomes, were found to have log-linear kinetics, suggesting that one-compartment model could account for the pharmacokinetic mechanism for the liposomes. This was similar to previously published studies [Hong et al., Clin Cancer Res; 5: 3645-3652, 1999; Allen, T. M., Trends Pharmacol Sci; 15: 215-220, 1994]. FIG. 3B shows that ¹¹¹In-NanoVNB has a different pharmacokinetic characteristic compared to ¹¹¹In-Lipo-DTPA and ¹⁷⁷Lu-Lipo-DTPA or previous studies of conventional liposome. Log-linear kinetics were found with good fit before 36 hours, however, no significant elimination was noted thereafter. One possible explanation is that equilibrium established between elimination from reticulum endothelium system (RES) and other liposome reservoirs after 36 hours. When the serum concentration of liposomes had decreased to 5 orders, liposomes released from other reservoirs may play a role to maintain stable serum concentration.

FIG. 3C illustrates the plasma concentration-time profile for 40 μCi of In-oxine (6% PEGDSPE-NanoVNB labeled with ¹¹¹In-oxine), ¹¹¹In-iono-PEG (6% PEGDSPE-NanoVNB labeled with ¹¹¹In-ionophore) and ¹⁷⁷Lu-iono-PEG (6% PEGDSPE-NanoVNB labeled with ¹⁷⁷Lu-ionophore) from an in vivo blood clearance study in normal BALB/c mice. The three tested liposomes all behaved as steric stabilized liposomes and showed similar pharmacokinetic characteristic as that for ¹¹¹In-Lipo-DTPA and ¹¹¹In-NanoVNB.

Results described above demonstrated the long-circulating pharmacokinetic characteristic of a liposome composition according to various embodiments of the present invention. Such pharmacokinetic characteristic does not depend on the particular loading component, i.e., the active encapsulated agent, such as ¹¹¹In-oxine, ¹¹¹In-ionophore or ¹⁷⁷Lu-ionophore.

SPECT Imaging of ¹¹¹In-Liposome

When the tumor nodule was induced, the animals were subject to SPECT imaging after the radiolabeled liposomes i.v. administration. The representative images of the HT-29 tumor-bearing animals are shown in the FIGS. 4A through to 4D. As shown in FIG. 4A, the tumor/muscle (T/M) ratios were (A) 4.39 for the HT-29 tumor-bearing mouse injected with 10 mg/kg ¹¹¹In-VNB-liposome, and (B) 2.25 for the HT-29 tumor-bearing mouse injected with 5 mg/kg ¹¹¹In-VNB-liposome. As shown in FIG. 4B through to 4D, each mouse was injected with 100 μCi ¹¹¹In-DTPA-liposome, wherein ‘lv’ stands for liver, ‘sp’ stands for spleen and ‘T’ stands for ‘tumor’. The images were typical of long-circulating liposomes containing PEGylated lipid. The liver was the organ with the highest amount of radioactivity, which continued to increase up to 72 h. In tumor-bearing animals, significant radioactivity also accumulated in the tumor nodule region. The image contrast was deemed sufficient for diagnostic imaging 4 hours postinjection. However, images obtained at 24 and 48 hours postinjection were sharper in terms of target tumor to background contrast as evident in FIGS. 4A through to 4D. The enhancement of target imaging over time may be caused by more background activity clearance or due to increased accumulation of liposomes in target region with time.

Whole-Body-Auto-Radiography (WBAR) Imaging

FIG. 5 shows the results of the WBAR imaging. Gray-scale photos of the anatomy were provided side-by-side to WBARs. The tumor volume was reduced from 198.7 mm³, to 189.3 mm³, and further to 53.3 mm³, when the concentration of ¹¹¹In-VNB-liposome was increased from 0 mg/kg, to 5 mg/kg, and further to 10 mg/kg, respectively. The WBARs and the gray-scale digital photos were taken 29 days post inoculation of the tumor cells, wherein ‘br’ stands for brain, ‘1 g’ stands for lung, ‘lv’ stands for liver, ‘sp’ stands for spleen, ‘br’ stands for bone marrow and ‘kd’ stands for kidney. Liposomes accumulate preferentially in the liver and the tumor tissue, because of the locally altered physiology characterized by enhanced blood flow and vascular permeability, and influx of macromolecules into the tumor nodule. The migration of liposomes into the tumor region through the leaky vascular endothelium is very similar to enhanced permeability and retention effect observed in tumor vasculature. Thus, liposomes passively target the tumor nodule region. Indeed, as shown in FIG. 4D, the mouse with tumor-bearing nodule accumulated less ¹¹¹In-Lipo-DTPA in several organs, except liver, spleen and tumor.

Results described above demonstrated that liposome compositions according to embodiments of the present invention can be used successfully for tumor targeted distribution of an imaging agent. Such composition can thus be used in nuclear imaging for in vivo cancer diagnostics.

Therapeutic Efficacy

To study the therapeutic efficacy of a liposome composition according to embodiments of the invention, SCID mice were inoculated with 2×10⁶ HT-29/luc tumor cells. Starting on the 20^{th} day after tumor cell inoculation, the volume of the tumor in the mice was monitored twice a week thereafter, using bioluminescence imaging (BLI) and Caliper measurements. Also starting on the 20^{th} day after tumor cell inoculation, ¹¹¹In-NanoVNB was injected intravenously to the mice for tumor treatment.

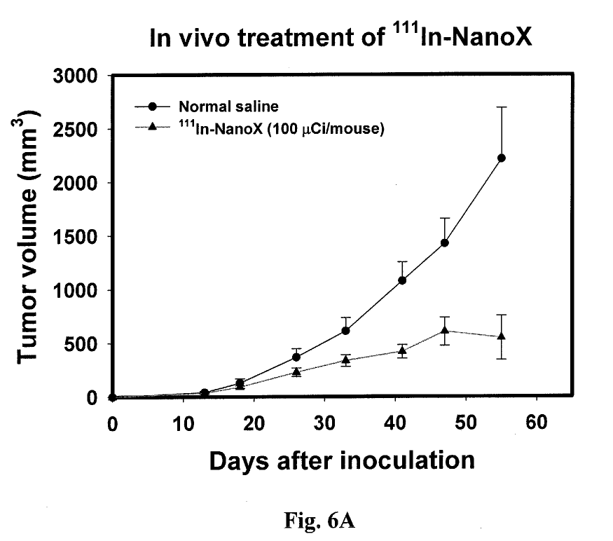

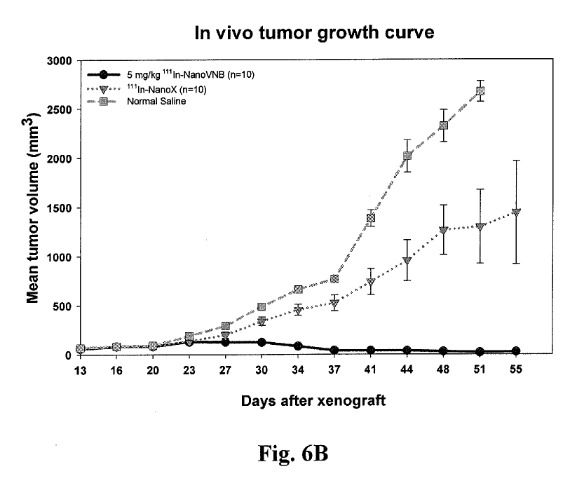

FIG. 6A and FIG. 6B illustrate the therapeutic efficacy of passively targeted radiotherapeutic agent, ¹¹¹In-NanoX, which comprises auger electron radionuclide payloads as compared with a normal saline control. As shown in FIG. 6A, effective inhibition of tumor growth was found by ¹¹¹In-liposome (¹¹¹In-NanoX, 100 μCi×4). And data were shown as mean±S.E. of five mice. FIG. 6B revealed the combination or additive therapeutic effect of passive targeted bimodality radiochemotherapeutic agent, ¹¹¹In-NanoVNB (¹¹¹In; 100 μCi×4, VNB:5 mg/kg×4). The results shown in FIGS. 6A and 6B were obtained from Caliper assay. As shown in FIGS. 6A and 6B, effective inhibition of tumor growth was found by ¹¹¹In-NanoVNB (¹¹¹In-VNB-liposome, 5 mg/kg and 100 μCi)ₓ₄ and ¹¹¹In-NanoX (¹¹¹In-liposome, 100 μCi)×4. Similar results were also observed using in vivo optical BLI assay. The efficacy of ¹¹¹In-NanoX with auger electron radiotherapy and efficacy of ¹¹¹In-NanoVNB with radiochemotherapy formulated using the most stable liposome formulation, TEA-SOS-loaded liposomes, were studied. The efficiency in suppressing the tumor growth of ¹¹¹In-NanoX was shown to be considerably better than control group by accumulated auger electron radionuclide in tumor site as shown in FIGS. 6A and 6B. The ¹¹¹In-NanoVNB was shown to be considerably more efficacious in suppressing the growth of HT-29 tumors that free vinorelbine combined with auger electron radionuclide, causing tumors to regress, while in the control group the tumors always continued to grow as evident in FIG. 6B. There was tolerable change in the animals' body weight during the course of treatment indicating the treatment was well tolerated.

The Antitumor Efficacy of Synergistic Radiochemotherapy by ¹¹¹In-VNB-Liposome in Syngeneic C26 Colon Carcinoma Model

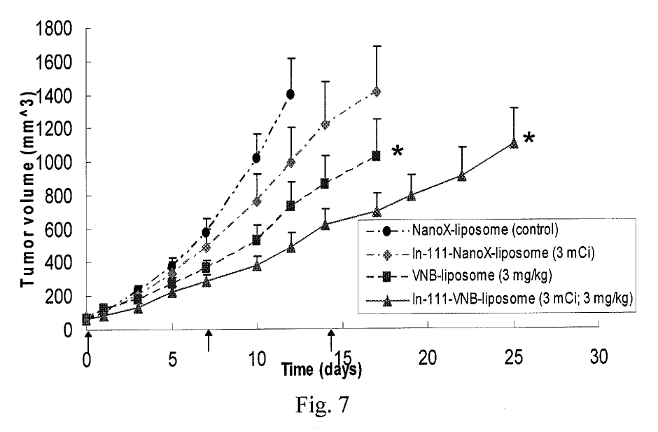

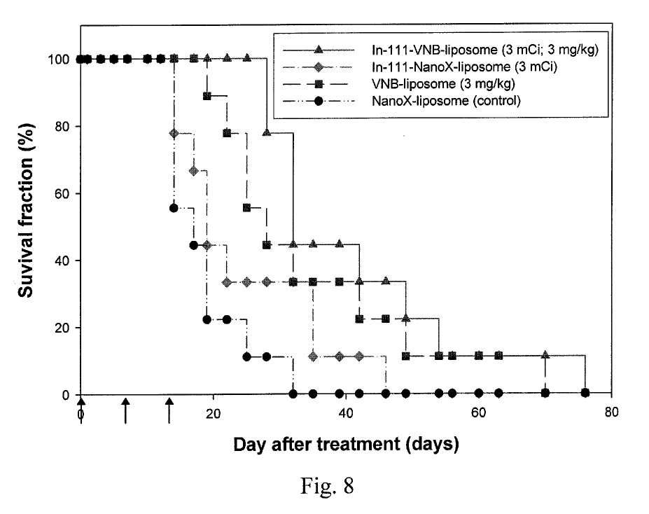

The synergistic antitumor efficacy of ¹¹¹In-VNB-liposome (¹¹¹In-NanoVNB) formulated using TEA-SOS-loaded liposomes was also studied in a multi-dose synergistic colon carcinoma model (C26). The liposomes were prepared with 0.9 mol % PEG-DSPE and were loaded at a VNB-to-PL ratio of 350 g VNB/mol PL and ¹¹¹In-to-vesicle ratio of 0.5 to 1 ¹¹¹In per vesicle. In FIG. 7, the tumor-bearing mice (n=9 for each group, tumor volume about 75 mm³) were injected intravenously with NanoX (), ¹¹¹In-NanoX (♦), VNB-liposome (▪) or ¹¹¹In-VNB-liposome (▴) at 0, 7, and 14 days. The zero time point indicates the initiation of therapy. SEM. *: p<0.05 compared with control group. The ¹¹¹In encapsulated liposome (¹¹¹In-liposome) at 3 mCi and liposomal vinorelbine (VNB-liposome) at 3 mg/kg, was considerably efficacious in reducing the tumor growth than control group as shown in FIG. 7. FIG. 8 showed that the treated mice had a significantly prolonged survival compared with the controls. The ¹¹¹In-NanoVNB was shown to be considerably more efficacious in suppressing the growth of C26 tumors and the synergistic antitumour action has also been demonstrated that free vinorelbine combined with auger electron radionuclide, causing tumors to regress, while in the ¹¹¹In-liposome and VNB-liposome groups the antitumor efficacy were less effect than ¹¹¹In-VNB-liposome group (FIGS. 7 and 8). There was tolerable change in the animals' body weight during the course of treatment indicating the treatment was well tolerated. Due to the rapid growth rate of these tumors, it is possible that this improvement may prove to be even more substantial when ¹¹¹In-VNB-liposome is tested in slower growing tumors. It is surprising to find that the group of ¹¹¹In-liposome only (without VNB) was effective in tumor reduction (FIGS. 6A, 6B, 7 and 8), suggesting, that auger electron may also play a role in the therapeutic efficacy. However, the synergistic effect was found with groups of ¹¹¹In combined with VNB-liposome.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims. All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A radiolabeled liposome comprising:
a) a liposome composition having a particle forming component and an agent-carrying component enclosed by the particle forming component; and
b) a radiolabeled agent entrapped within the liposome composition, wherein the radiolabeled agent comprises a radionuclide selected from the group consisting of ¹¹¹In, ¹⁷⁷Lu, ⁹⁰Y, ²²⁵Ac, and their daughter radionuclides.

2. The radiolabeled liposome of claim 1 further comprising an antineoplastic agent entrapped within the liposome composition.

3. The radiolabeled liposome of claim 1, wherein
a) the particle forming component comprises a phospholipid or a derivative thereof, and polyethylene glycol (PEG) or a derivative thereof;
b) the agent-carrying component comprises a chelator selected from the group consisting of ethylenediaminetetraacetic acid (EDTA), diethylenetriaminepentaacetic acid (DTPA), 1,4,7,10-tetraazacyclododecane-1,4,7,10-tetraacetic acid (DOTA), nitroltriacetic acid (NTA), deferoxamine, and dexrozpxane; and
c) the radiolabeled agent is ¹¹¹In or ¹⁷⁷Lu.

4. The radiolabeled liposome of claim 1, wherein
a) the particle forming component comprises a phospholipid or a derivative thereof, and polyethylene glycol (PEG) or a derivative thereof;
b) the agent-carrying component is selected from the group consisting of sulfate salt, polysulfate salt, phosphate salt, and polyphosphate salt; and
c) the radiolabeled agent is ¹¹¹In or ¹⁷⁷Lu.

5. The radiolabeled liposome of claim 3 further comprising an effective amount of vinorelbine entrapped within the liposome composition.

6. The radiolabeled liposome of claim 3, wherein the liposome has a mean particle diameter of about 30 nm to about 200 nm.

7. A kit for targeting a radiolabeled agent to a tumor site in a subject in need thereof, the kit comprising:
a) a liposome composition comprising:
i) a particle forming component comprising a vesicle-forming lipid from a group of amphipathic lipids having hydrophobic and polar head group moieties alone or in combination;
ii) an agent-carrying component enclosed by the particle forming component, wherein the agent-carrying component has a chemical entity that contains one or more negatively charged groups or trapping ions; and
iii) a radiolabeled agent entrapped within the liposome composition via an electrostatic charge-charge interaction with the agent-carrying component, wherein the radiolabeled agent comprises a radionuclide selected from the group consisting of ¹¹¹In, ¹⁷⁷Lu, ⁹⁰Y, ²²⁵Ac and their daughter radionuclides; and
b) an instruction manual.

8. The kit of claim 7, wherein the radiolabeled agent further comprises an antineoplastic agent.

9. A method for preparing a radiolabeled liposome comprising:
a) providing a liposome composition comprising a particle forming component and an agent-carrying component enclosed by the particle forming component; and
b) entrapping a radiolabeled agent within the liposome composition, wherein the radiolabeled agent comprises a radionuclide selected from the group consisting of ¹¹¹In, ¹⁷⁷Lu, ⁹⁰Y, ²²⁵Ac, and their daughter radionuclides.

10. The method of claim 9, wherein
a) the liposome composition comprising a phospholipid or a derivative thereof, and polyethylene glycol (PEG) or a derivative thereof, and a chelator selected from the group consisting of ethylenediaminetetraacetic acid (EDTA), diethylenetriaminepentaacetic acid (DTPA), 1,4,7,10-tetraazacyclododecane-1,4,7,10-tetraacetic acid (DOTA), nitroltriacetic acid (NTA), deferoxamine, and dexrozpxane; and
b) the radiolabeled agent is ¹¹¹In-oxine, ¹¹¹In-ionomycin, ¹⁷⁷Lu-oxine or ¹⁷⁷Lu-ionomycin.

11. A method for diagnosing and treating a tumor in a subject comprising:
a) providing a liposome composition having a particle forming component, an agent-carrying component and a radiolabeled agent, wherein the agent-carrying component and the radiolabeled agent are enclosed by the particle forming component, and the radiolabeled agent comprises a radionuclide selected from the group consisting of ¹¹¹In, ¹⁷⁷Lu, ⁹⁰Y, ²²⁵Ac and their daughter radionuclides; and
b) administering the liposome composition to the subject.

12. The method of claim 11, wherein the liposome composition is administered intravenously or intraperitoneally.

13. The method of claim 11, wherein the liposome composition further comprises an antineoplastic agent entrapped within the liposome composition.

14. The method of claim 11 further comprising measuring or detecting the amount of radiation emitted from the radionuclide.

15. A nanoparticle comprising a radionuclide selected from the group consisting of ¹¹¹In, ¹⁷⁷Lu, ⁹⁰Y, ²²⁵Ac, and their daughter radionuclides.

16. The nanoparticle of claim 15 further comprising an antineoplastic agent.

17. The nanoparticle of claim 16, wherein the antineoplastic agent is selected from the group consisting of a vinca derivative drug, vinorelbine, vincristine, vinblastine, vinflunine, an anthracycline drug, doxorubicin, daunorubicin, mitomycin C, epirubicin, pirarubicin, rubidomycin, carcinomycin, N-acetyladriamycin, rubidazone, 5-imidodaunomycin, N-acetyldaunomycine, daunoryline, mitoxanthrone, a camptothecin compound, camptothecin, 9-aminocamptothecin, 7-ethylcamptothecin, 10-hydroxycamptothecin, 9-nitrocamptothecin, 10,11-methylenedioxycamptothecin, 9-amino-10,11-methylenedioxycamptothecin, 9-chloro-10,11-methylenedioxycamptothecin, irinotecan, topotecan, lurtotecan, silatecan, (7-(4-methylpiperazinomethylene)-10,111-ethylenedioxy-20(S)-camptothecin, 7-(4-methylpiperazinomethylene)-10,11-methylenedioxy-20(S)-camptothecin, 7-(2-N-isopropylamino)ethyl)-(20S)-camptothecin, an ellipticine compound, ellipticine, 6-3-aminopropyl-ellipticine, 2-diethylaminoethyl-ellipticinium and salts thereof, datelliptium, and retelliptine.

18. A method of treating a tumor in a subject comprising:
a) administering to the subject at a tumor site a long-circulating nanoparticle comprising a heavy element combined with an antineoplastic agent, wherein the heavy element is selected from the group consisting of ¹¹¹In, ¹⁷⁷Lu, ⁹⁰Y, ²²⁵Ac and their daughter radionuclides; and
b) irradiating the tumor site.
